(19) United States
(12) Patent Application Publication
(10) Pub. No.: US 2009/0259137 A1
(43) Pub. Date: Oct. 15, 2009

(54) DETERMINATION OF BIOSENSOR CONTACT QUALITY

(75) Inventors: Emir Delic, Lidcombe (AU); Bashir Fouad Ziady, San Francisco, CA (US); Marco Kenneth Della Torre, Mortdale (AU); Geoffrey Ross MacKellar, Elanora Heights (AU)

Correspondence Address: FISH & RICHARDSON P.C., PO BOX 1022, MINNEAPOLIS, MN, 55440-1022, US

(73) Assignee: Emotiv Systems Pty Ltd

(21) Appl. No.: 12/270,739

(22) Filed: Nov. 13, 2008

Related U.S. Application Data
(60) Provisional application No. 60/988,075, filed on Nov. 14, 2007.

(51) Int. Cl. A61B 5/0482; A61B 5/0478; A61B 5/053

(52) U.S. Cl. 600/545; 600/547

(57) ABSTRACT

A method of assisting a user in bringing a plurality of EEG electrodes into proper contact with the user's scalp includes receiving a signals from EEG electrodes, determining a state of contact with the user's scalp, and displaying a representation of the user's scalp and an indicator of the state of contact, the indicator positioned on the representation in a position representative of a position of an associated electrode on the user's scalp. Determining quality of contact of an electrode on a user can includes applying a reference signal to a user, receiving a measured signal from an electrode for monitoring bioelectric signals of the user, and determining a state of contact of the electrode with the user based on the measured signal and the reference signal.

### RELATED APPLICATIONS

This application claims the benefit under 35 U.S.C. § 119(e) of U.S. Provisional Patent Application Ser. No. 60/988,075, filed on Nov. 14, 2007.

### TECHNICAL FIELD

This disclosure relates generally to interaction with machines using bio-sensing.

### BACKGROUND

Interactions between humans and machines are usually restricted to the use of input devices such as keyboards, joy sticks, mice, trackballs and the like. Such input devices are cumbersome because they must be manually operated, and in particular operated by hand. Some input devices have been developed to detect eyeball movement or are voice activated to minimize the physical movement required by a user in order to operate these devices.

One area that has been under investigation is the use of bioelectric signals, such as electroencephalograph (EEG) signals, to control machines. Such investigations have generally been limited to clinical and experimental situations. In a clinical application, the electrodes are applied to a patient by a relatively skilled technician. In addition, a patient in a clinical situation is more likely to be tolerant of some level of discomfort or inconvenience when testing and calibrating electrodes than a person in a non-clinical setting.

In general, bio-signal sensing electrodes, particular passive electrodes, are prone to noise and can require noise canceling techniques to achieve satisfactory performance. One noise canceling technique, to minimize impedance at the skin-electrode interface and to minimize interference, involves conditioning the skin where the electrode is to be applied. Typically a scalpel is used to scrape the skin and a liquid disinfectant solution is used to clean the area. Another approach to minimizing impedance and interference at the skin-electrode interface, commonly combined with abrasive and depilatory preparation, is to fill any gap at the interface with a conductive gel or saline solution that can regulate the impedance.

A conventional apparatus for applying electrodes to a subject's head includes a flexible cap that covers the subject's entire scalp and includes a strap beneath the chin, so that the cap may be snugly secured to the subject's head. This type of apparatus is typically used in a clinical setting and can include over 100 electrodes for some applications.

### SUMMARY

In one aspect, a method of assisting a user in bringing a plurality of EEG electrodes into proper contact with the user's scalp includes receiving a plurality of signals from the plurality of EEG electrodes, determining from the plurality of signals a state of contact with the user's scalp for each of at least two of the plurality of electrodes, and displaying on a display a representation of the user's scalp and an indicator of the state of contact for each of the at least two of the plurality of electrodes, each indicator positioned on the representation in a position representative of a position of an associated electrode on the user's scalp.

Implementations may include one or more of the following. Displaying the indicator may include selecting one of a plurality of predetermined colors based on the state of contact, and displaying the color in the indicator. Selecting a color may include selecting green if the state of contact is determined to be satisfactory, or selecting red if the state of contact is determined to be unsatisfactory. The indicator may be a shape, e.g., a circle, filled with the selected color. Determining the state of contact may includes determining a numerical indicator of the state of contact, and displaying the indicator may includes displaying the numerical indicator. The numerical indicator may be between 0 and 1. The indicators may be non-overlapping.

In another aspect, a method of determining quality of contact of an electrode on a user includes applying a reference signal to a user, receiving a measured signal from an electrode for monitoring bioelectric signals of the user, and determining a state of contact of the electrode with the user based on the measured signal and the reference signal.

Implementations may include one or more of the following. Determining the state of contact may include comparing the measured signal to the reference signal. Comparing the measured signal to the reference signal may include dividing an amplitude of the measured signal by an amplitude of the reference signal to generate a normalized amplitude. The reference signal may have a frequency greater than 100 Hz, e.g., a frequency between 200 and 300 Hz. The reference signal may have an amplitude of 10 uA or less. Signals having the frequency may be filtered from the measured signal to generate a filtered signal and the filtered signal may be directed to a state detection engine. Determining the state of contact may include comparing the normalized amplitude to a threshold. Determining the state of contact includes determining that the state of contact is satisfactory if the normalized amplitude is greater than a value between about 0.7 and 0.9, e.g., 0.8. Determining the state of contact may include comparing the normalized amplitude to a plurality of thresholds. An indicator of the state of contact may be displayed on a display. A representation of the user's scalp may be displayed on the display, and the indicator may be positioned on the representation in a position representative of a position of the electrode on the user's scalp.

Implementations can realize one or more of the following advantages. An EEG headset can be placed on the head and used more easily in a non-clinical application. For example, the EEG headset with electrodes can be applied by a user to the user's own scalp. The EEG headset can be placed by a person with no training or knowledge of correct application or placement of the electrodes, and a suitable signal is more likely to be generated. Such a headset can use low-cost passive electrodes. It is not necessary to prepare the skin or apply liquid saline, oil or water-based contact gel, to obtain a suitable signal. The determination of contact quality can be more reliable across a population of users. Feedback of the contact quality for each individual biosensor can be provided to the end user or an automated mechanism for compensating for a deteriorating signal.

The details of one or more embodiments are set forth in the accompanying drawings and the description below. Other features, objects, and advantages will be apparent from the description and drawings, and from the claims.

### DESCRIPTION OF DRAWINGS

FIG. 1 is a schematic representation of an example signal acquisition system.

FIG. 2 is a side view of an example headset on a subject's head.

FIG. 3 is a flow chart illustrating an exemplary method of signal acquisition.

FIG. 4 is a circuit diagram of a signal quality detection circuit.

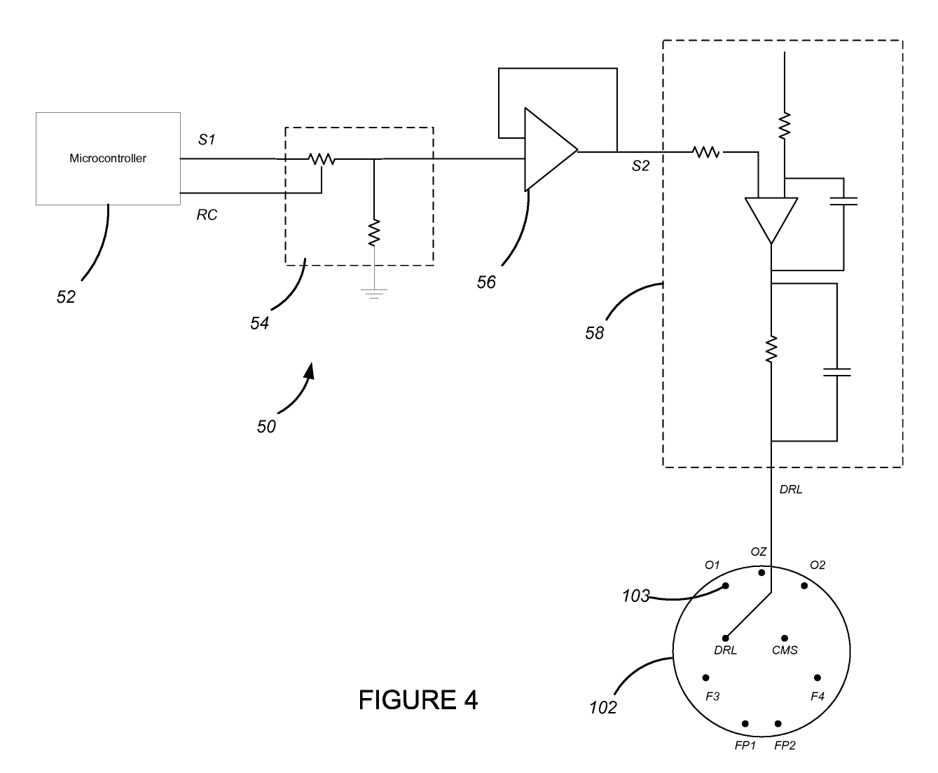

FIG. 5 illustrates a graphical user interface to aid the subject during placement of an electrode headset.

Like reference symbols in the various drawings indicate like elements.

### DETAILED DESCRIPTION

An electrode system to capture bioelectric signals from a subject generally should address various requirements including safety needs, cost, power consumption, performance, ease of use and subject comfort. However, the relative importance of these factors may be somewhat different in a non-clinical application than in a clinical application. In particular, in a non-clinical application, the electrodes are more likely to be applied by a person with no training or knowledge of correct application or placement of the electrodes. In addition, the user may be attempting to apply the electrodes to himself or herself. Consequently, it is more likely that the electrodes will not be properly placed, both in terms of location and necessary contact pressure or contact area on the subject's skin, e.g. the scalp in the case of EEG. As a result, signal noise becomes more likely, and the analysis of the signals from the electrodes becomes more prone to error.

In general, the lack of training or knowledge of the user can be alleviated by providing a system that automatically determines whether signal-quality for individual electrodes is satisfactory, and displays this information to the user. Thus, the display effective acts as a feedback mechanism to inform the inexperienced user of incorrect positioning of the electrodes. The user can then reposition or adjust the pressure on the electrodes indicated as being improperly placed until an acceptable level of contact to the user's scalp is achieved.

If the contact quality deteriorates over time, as the headset is being used, the user can be informed that a particular electrode needs readjustment. For example, dry sensors might perform worse as the skin dries out, whereas wet sensors might lose conductivity as the electrolyte in the contact material is depleted. By alerting the user of changes in the contact quality, the user can take corrective action.

In addition, information regarding the contact quality can be directed to an automated mechanism for compensating for a deteriorating signal. For example, in the case of a wet sensor, the system might include automatic control for replenishing the conductive solution that is triggered as the contact quality deteriorates.

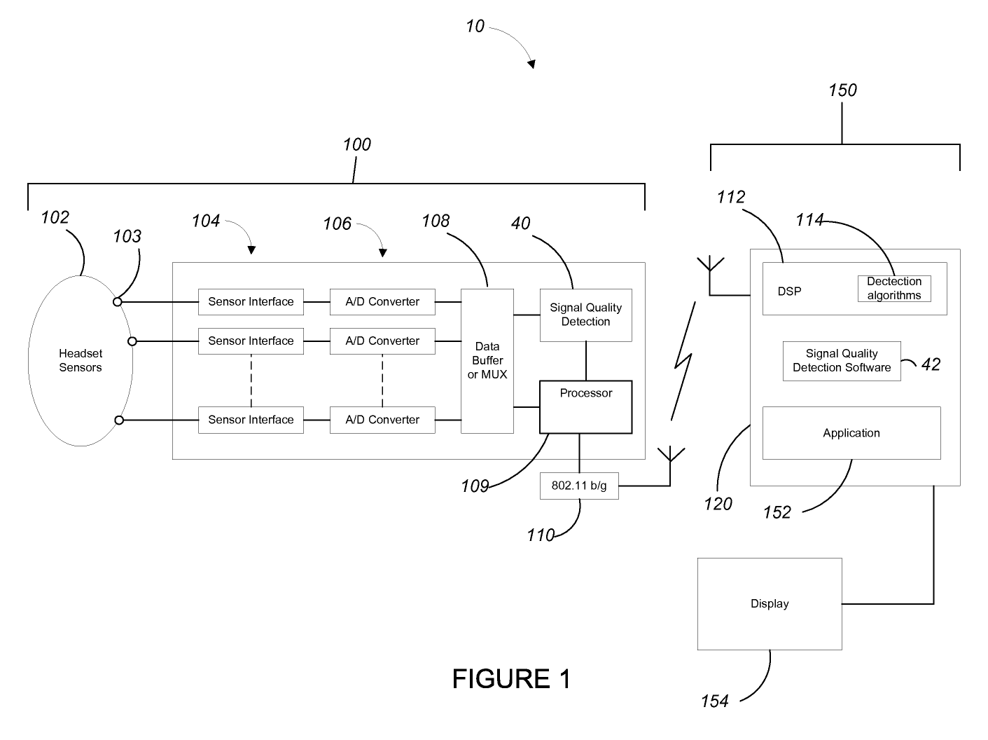

Turning now to FIG. 1, there is shown a system for detecting and classifying mental states and facial expressions (collectively simply referred to as “states”) of a subject and generating signals to represent these states. In general, the system can detect both non-deliberative mental states, for example emotions, e.g., excitement, happiness, fear, sadness, boredom, and other emotions, and deliberative mental states, e.g., a mental command to push, pull or manipulate an object in a real or virtual environment. The system is capable of detection of mental states (both deliberative and non-deliberative) and facial expressions using solely electrical signals, particularly EEG signals, from the subject, and without direct measurement of other physiological processes, such as heart rate, blood pressure, respiration or galvanic skin response, as would be obtained by a heart rate monitor, blood pressure monitor, and the like.

Signals from the system representing the state of the user are directed to an application 152. The application 152 can also respond to input events by modifying an environment, e.g., a real environment or a virtual environment as displayed on a display 154. Thus, the mental state or facial expressions of a user can be used as a control input for a gaming system, or another application (including a simulator or other interactive environment).

The system includes two main components, a neuro-physiological signal acquisition device 102, e.g., an electrode headset, that is worn or otherwise carried by a subject, and a state detection engine 114. In brief, the neuro-physiological signal acquisition device 102 detects bio-signals from the subject, and the state detection engine 114 implements one or more detection algorithms that convert these bio-signals into signals representing the presence (and optionally intensity) of particular states in the subject. In addition, an automated signal quality detection process 40 detects the quality of the bio-signals from the signal acquisition device 102, and user interface software 42 can display indicia of the signal quality to the user.

As a headset, the neuro-physiological signal acquisition device 102 includes multiple electrodes 103 which, when the headset is properly placed on the subject's head, electrically contact the subject's scalp at predetermined locations to measure EEG signals. It should be noted, however, that the EEG signals measured and used by the system 10 can include signals outside the frequency range, e.g., 0.3-80 Hz, that is customarily recorded for EEG. Moreover, the system 10 can utilize even higher frequencies, e.g., above 100 Hz, for determination of the sensor contact quality. Unlike systems that provide high-resolution 3-D brain scans, e.g., MRI or CAT scans, the headset is generally portable and non-constraining.

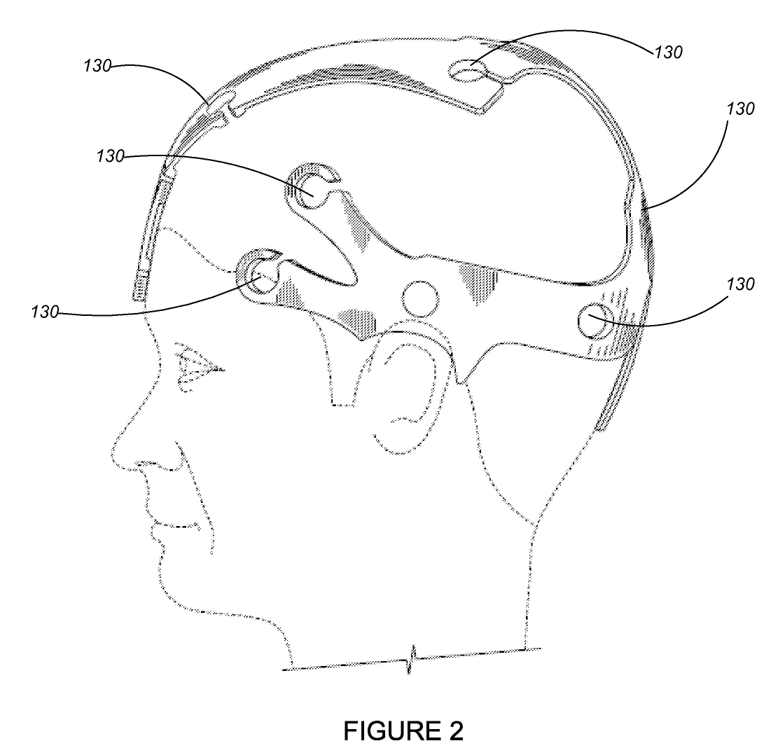

Referring to FIG. 2, one implementation of an electrode headset 102 is shown. The electrode headset 102 is configured to fit snugly on a subject's head. The headset 102 includes multiple electrode mounts, each configured to mount an electrode. In this implementation the electrode mounts are apertures 130 configured to receive and mount an electrode, e.g., by a press fit (the electrodes themselves are not illustrated in FIG. 2 so as to more clearly illustrate the apertures 130). However, it should be noted that other configurations of electrode mounts can be used. For example, an electrode can be mounted to the electrode headset using a clamp, screw or other suitable connection mechanism and/or configuration.

In general, the headset 102 can be sufficiently flexible to fit comfortably on the subject's head, but sufficiently rigid to hold the electrodes 103 in approximately the correct positions on the scalp.

Returning to FIG. 1, a headset assembly 100 includes the headset 102 itself, the electrodes 103, and additional circuitry for transmitting the signals from the headset to the state detection engine 114. The signals detected by each of the electrodes 103 on the headset 102 are fed through a sensory interface 104, which can include an amplifier to boost signal strength and a filter to remove noise, and then digitized by an analog-to-digital converters 106. Digitized samples of the signal captured by each of the scalp sensors can be stored in a data buffer 108. The data buffer 108 is connected to the signal quality detection circuitry 40.

The data buffer is connected, e.g., through an input/output transmission device 110, such as a wireless 2.4 GHz device, a WiFi or Bluetooth device, to a processing system 120 that runs the state detection engine 114. A microcontroller 109 controls communication between the memory 108 and the input/output device, and handles other aspects of the headset assembly 100, such as controlling power to components to conserve battery lifetime. In particular, the microcontroller 109 can respond to queries regarding signal quality from the user interface 42 or other components of the processing system 120 by accessing the signal quality detection circuitry 40, and returning the signal quality data.

The processing system 120 can include a digital signal processor (DSP) 112, to perform desired functional steps of the state detection engine. In general, the DSP 112 performs preprocessing of the digital signals to reduce noise, transforms the signal to “unfold” it from the particular shape of the subject's cortex, and performs the emotion, deliberative mental state and facial expression detection algorithms on the transformed signal. Although illustrated as part of a DSP, the state detection engine can be implemented primarily in hardware using, for example, hardware components such as an Application Specific Integrated Circuit (ASIC), as software, for example, as a memory including a series of instructions to be performed by a DSP or general purpose computer, or using a combination of both software and hardware.

Systems for detecting mental states are described in U.S. Patent Publication No. 2007-0173733 and U.S. Patent Publication No. 2007-0066914, both of which are incorporated by reference. Systems for detecting facial expressions are described in U.S. Patent Publication No. 2007-0179396, which is incorporated by reference.

The processing system 120 can include the user interface software 42 to display the indicia of the signal quality to the user on the display 154.

In the illustrated implementation, the head set assembly 100 includes the head set 102, interface 104 and A/D converter(s) 106, MUX/data buffer 108, microcontroller 109, wireless transmission device 110, and signal quality detection circuitry 40, as well as a battery for power supply. In addition, in the illustrated implementation, application 152 and the DSP 112 are part of the same external device 150, e.g., a general purpose computer or a game console.

However, many other configurations are possible. The state detection engine 114 can be in a dedicated processor unit that is separate from the platform 150 running the application 152. In this case, the processor unit can includes the wireless receiver to receive data from the headset assembly. The processor unit can be connected to the platform 150 by a wired or wireless connection, such as a cable that connects to a USB input of the platform 150. The state detection engine 114 can be software running on the same processor as the application 152. Various components, can be moved onto or off the headset assembly. For example, the signal quality detection circuitry 40 could be part of a unit separate from the platform 150 running the (e.g., the same unit with the state detection engine 114), or be integrated into the platform 140. The buffer 108 could be eliminated or replaced by a multiplexer (MUX), and the data stored directly in the memory of the processing system. A MUX could be placed before the A/D converter stage so that only a single A/D converter is needed. The connection between the head set assembly 100 and the platform 150 can be wired rather than wireless.

As noted above, an automated signal quality detection process 40 detects the quality of the bio-signals from the signal acquisition device 102, and the user interface software 42 displays indicia of the quality on a display 154.

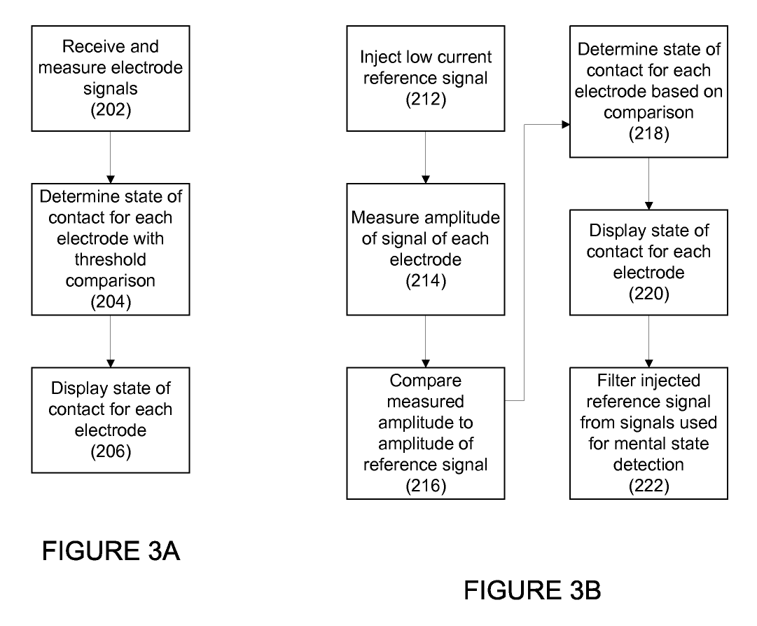

Referring to FIG. 3A, in a first implementation, when the headset is placed on the user's head, the user can initiate or the system can auto-initiate a signal quality feedback mode. The signal from each electrode is measured (step 202), and a state of contact is calculated for each electrode from one or more properties of the signal (step 204). The state of contact for each electrode can be determined using threshold based classifier system using factors such as amplitude, frequency and transient information of the signal of that electrode. Such a threshold based classifier system can calculate a weighted sum of the amplitude, frequency and transient information of the signal, and compare the weighted sum to a predetermined threshold(s). If the weighted sum is outside the accepted threshold(s), this indicates an un-acceptable state of contact. The state of contact for each electrode is then displayed to the user on a graphical interface, e.g., a monitor 154 (step 206). It is also possible to have multiple thresholds that provide a range of contact quality states. The range of contact quality states can show intermediate levels of contact that might prompt some kind of action by the user, e.g., replacing or re-hydrating the sensor contact material as it is drying out, in case of conductive felt tip based sensor.

There are potential problems with this approach. If the characteristics of the hardware acquisition system, sensors or other parts in the signal chain are changed, then the algorithm used by the threshold based classifier system might need to be retuned. In addition, there are large variations in skin impedances between individuals, making this threshold based approach less reliable across a population. The threshold based implementation can also introduce significant processing latencies that can be evident to the user as fixed delays in graphical representation of signal quality. For example, if a particular sensor becomes mechanically offset or the noise increases, the threshold based implementation may have a lag before this information is displayed to the user. Finally, this technique is sensitive to facial expression or muscle artifacts, which might falsely trigger signal quality algorithms.

Referring to FIG. 3B, in a second implementation, a low current, low voltage, reference signal is injected into the user, i.e., applied to one of the electrodes (step 212). The current of the reference signal can be limited to 10 microamperes or less to meet FDA standards and to make sure there is no discomfort to the user. This reference signal can be a regular waveform, e.g., a square wave or sine wave, with a frequency outside the standard EEG frequency range, but within the range detected by the acquisition system. For example, the reference signal can have a frequency greater than 100 Hz, e.g., more than 200 Hz. However, some implementations can utilize a reference signal that is inside of the EEG band.

In some implementations, the reference signal is combined with a biasing signal and injected into the user through a driven right leg (DRL) electrode on the headset 102. The DRL electrode can be one of the biosensor electrodes 103. A potential advantage of the described implementation is that it uses existing circuitry, and does not require an accurate measurement of the contact impedance. Alternative implementations can use an injection electrode separate from the biosensor electrodes 103.

In some implementations, scalp impedance can be detected. In this case a constant current source circuit can be used to generate the injected signal.

The injected reference signal is effectively combined with EEG signals and other artifacts in the scalp, before its picked up by the EEG sensors. Each EEG sensor will pick up a signal that is a linear combination of the injected reference, EEG, muscle artifacts and noise. The amplitude and phase of the input reference signal will be modified by the scalp tissue.

The amplitude of the injected signal will be attenuated at various stages along the path from the DRL electrode to the sensor electrode. The injecting and sensing electrodes, the electrode/scalp interface and the scalp tissue all play a part in the reference signal attenuation. The attenuation will become larger as the distance between the injection point and the sensing site increases. The major attenuation will happen at the sensor/scalp interface as the impedance of this site is the most significant in the chain. For this reason the system is relatively unaffected by absolute positioning of the injection electrode.

As described above, signals that are detected at various scalp locations will contain EEG data, artifacts, noise and injected reference signal components. As the frequency and phase characteristics of the injected reference signal are known, information about the amplitude of the reference signal component contained inside of the detected signal can be computed. This amplitude can then be compared to the amplitude of the reference signal before it was injected and thus the contact quality can be computed.

The reference signal can be a substantially spurious-free waveform, such as a clean sine wave. Such a waveform can add negligible harmonic distortion and can allow for simple filtering and removal after signal quality is determined. In general, however, any waveform of known amplitude and frequency can be used. It is possible to use other regular waveforms such as square waves, triangle waves, etc., that have different spectral characteristics, as long as care is taken not to inject unwanted harmonics into the EEG band of interest. In some implementations the reference signal is a square wave, as a square wave can be more easily generated in digital hardware. The reference signal can be generated inside of a microcontroller and its amplitude level adjusted through a fixed value resistor divider or an amplifier with a digitally controllable gain. More complex waveforms can be generated using analog oscillators, phase locked loops, or direct digital synthesis (DDS).

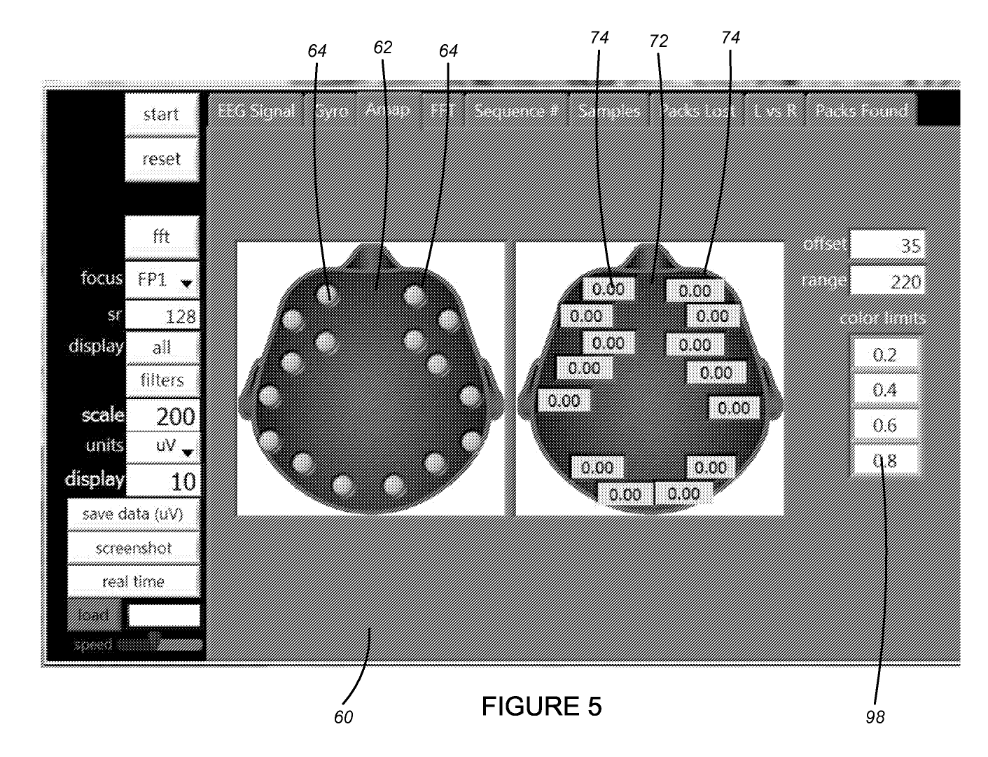

In one implementation of circuitry 50 for generating a reference signal is shown in FIG. 5. A precursor S1 to the reference signal is generated inside of microcontroller 54 (which can be the microcontroller 109). In this implementation the reference signal is a synchronized square wave at 273 Hz. This square wave is attenuated through an attenuator circuit 54, such as resistor divider, down to 100 uV. Optionally, the attenuator circuit 54 can be digitally controlled by a signal RC from the microprocessor 52. The signal is then buffered, e.g., by an op-amp buffer 56, to prevent output loading. The signal S2 is injected into the negative terminal of a driven right leg (DRL) amplifier 58. In essence, in this approach the DRL signal is modulated with square wave information. The DRL is responsible for biasing the subject's body to a reference potential in some EEG systems.

The sampling rate for the electrodes 103 can be 1024 samples per second, to ensure an accurate interpretation of the injected signal in the digital domain. Because the frequency of the reference signal is far away from the usable EEG band, it permits relatively easy and robust extraction before amplitude detection. In addition the harmonic distortion to the EEG signal from any injected signal is minimal.

For each sensor, the amplitude of the measured signal is determined in a narrow frequency band that matches the frequency of the reference signal (step 214). Thus, the system effectively measures the amplitude of the injected component at each of the sensors.

In one implementation, the amplitude of the square wave reference signal is detected using a derivative/zero-crossing based peak detection algorithm. Detected signals are first filtered with a 4th order band pass Infinite impulse response (IIR) filter with a pass band from 270 Hz to 276 Hz to remove all other frequencies that might affect the accuracy of the algorithm. The peak detection algorithm is then used to detect the amplitude of the square wave.

In alternative implementations, low frequency reference signals can be used, allowing for lower overall system sampling rates. In such implementations the amplitude of the injected reference signal should be lowered to keep the harmonic distortion in the EEG signal as low as possible.

Returning to FIG. 3B, the measured amplitude is then compared to the amplitude of the original injected reference signal (step 216).

The state of contact can then be determined for each electrode based on the comparison (step 218). If the contact is poor, then the measured amplitude will be low compared to the reference signal. In contrast, in the case of good contact, the measured amplitude will be high (approaching the amplitude of the original injected signal). In general, if there is no contact, then amplitude of the measured signal will be zero.

In particular, the measured amplitude can be normalized by dividing the measured amplitudes by the amplitude of the reference signal. For example, in one implementation the amplitude is divided by 100 uV (the original amplitude of injected signal) to generate the normalized value. This normalized amplitude can then be compared to threshold percentages to determine the state of contact of the electrode. For example, a normalized amplitude greater than 0.8 can indicate satisfactory contact. This method can be formed quickly and with high reliability to determine contact quality.

In addition, each electrode can be assigned one of a multiple of progressively superior contact states based on comparison to associated threshold ranges. For example, each electrode can be assigned one of four or more contact states, e.g., one of six or more contact states. For example, a normalized amplitude of 0 can indicate no contact, and normalized amplitudes less than 0.2, 0.2 to 0.4, 0.4 to 0.6, 0.6 to 0.8 and greater than 0.8 can indicate progressively superior contact states, with a normalized amplitude greater than 0.8 indicating good or satisfactory contact.

The signal quality for each electrode can be displayed to the user, e.g., on a video display (step 220). For example, the normalized value can be used to drive a color coded map of the users head where various colors represent different levels of sensor contact quality. The user can then reposition or adjust the pressure on the electrodes indicated on the display as having non-satisfactory contact until an acceptable level of contact to is achieved.

Referring to FIG. 5, a graphical user interface 60 can be displayed to aid the subject during placement of an electrode headset. The interface 60 includes a simplified schematic illustration 62 of a subject's head, and an indicator light 64 for each electrode. Each indicator light is positioned on the schematic 62 in a position generally equivalent to the position of the associated electrode on the subject's head when the headset is worn. The color of each indicator light 62 can indicate the contact status of the associated electrode. Thus, one of a plurality of predetermined colors can be selected based on the state of contact. In general, red can indicate unacceptable contact, whereas green can indicate acceptable contact. For example, a green light can indicate an acceptable level of contact (e.g., a normalized amplitude greater than 0.8 as discussed above), a red light can indicate lack of contact (e.g., a normalized amplitude of 0), and a yellow light can indicate partial but insufficient contact (e.g., a normalized amplitude greater than 0 but less than 0.8).

In addition to determining a state of contact of each sensor, the state of contact of the reference electrodes can also be measured. For example, when the headset is placed on the scalp and all of the sensors have a consistent low reading, it means that the reference signal is not being injected properly and that only a portion of it is getting through. This information can be displayed and the user can be prompted to adjust the reference sensor.

Similarly if the headset is sitting on the desk, the amplitude of the injected signals will be zero for all sensors. This information can be used to determine that the headset is not in use and can turn the headset OFF after some time period. In this way battery life can be extended.

Although FIG. 5 illustrates a circular indicator light, other indicators of the contact status can be used. For example, the indicator can be another simple shape, such a square, triangle or asterisk. In addition, although FIG. 5 illustrates a simplified top-down schematic view of the user's head, other representations of the user's scalp can be used. For example, the representation can include one or more side views. In addition, rather than a schematic, the representation can be an actual image of a person's head, e.g., the user's head. As another implementation, a representation of the headset can be displayed rather than a representation of the user's head.

Alternatively or in addition, the graphical user interface 60 can include a simplified schematic illustration 72 of a subject's head, and a numerical indicator 74 of the state of contact. The numerical indicator can be the normalized amplitude measurement.

The graphical user interface 60 can also include user editable fields 98 that permit the user to set the thresholds for the various states of contact.

Returning to FIG. 3B, before sending the measured biosignals to the state detection engine, the reference signal can be filtered out, leaving only the EEG information (step 222).

Using the feedback from the display, a user with no training or knowledge of correct application or placement of the electrodes should be able to place the headset properly on the user's head. Due to the use of the reference signal, determination of signal quality is less subject to artifacts or facial expressions and is generally independent of different skin types or other variances across populations, and is thus more reliable. The technique works in real time and can feedback information on signal quality to the user in a clear, concise, intuitive way.

In addition, the techniques is safe as the current injected is kept under 10 uA (existing medical instrumentation limit). Because the frequency of the injected waveform is outside of the conventionally utilized EEG range, it can be easily filtered out. The amplitude of the reference signal can be small enough so that harmonics of the waveform and beats with mains and sampling are below the noise floor.

Embodiments of the invention and all of the functional operations described in this specification can be implemented in digital electronic circuitry, or in computer software, firmware, or hardware, including the structural means disclosed in this specification and structural equivalents thereof, or in combinations of them. Embodiments of the invention can be implemented as one or more computer program products, i.e., one or more computer programs tangibly embodied in an information carrier, e.g., in a machine readable storage device or in a propagated signal, for execution by, or to control the operation of, data processing apparatus, e.g., a programmable processor, a computer, or multiple processors or computers. A computer program (also known as a program, software, software application, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file. A program can be stored in a portion of a file that holds other programs or data, in a single file dedicated to the program in question, or in multiple coordinated files (e.g., files that store one or more modules, sub programs, or portions of code). A computer program can be deployed to be executed on one computer or on multiple computers at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

A number of embodiments of the invention have been described. Nevertheless, it will be understood that various modifications may be made without departing from the spirit and scope of the invention. For example, another alternative implementation of the peak detection algorithm is based on a lock-in amplifier. In this implementation, the input signal from each sensor is band pass filtered and then multiplied by the reference signal. The resulting signal is low pass filtered. After some algebraic computation the output value is computed and represents the amplitude of the injected reference component inside of the input signal. The potential advantages of this approach are in lower computational requirements and higher accuracy in the presence of large background noise. Accordingly, other embodiments are within the scope of the following claims.

What is claimed is:

1. A method of assisting a user in bringing a plurality of EEG electrodes into proper contact with the user's scalp, comprising:
receiving a plurality of signals from the plurality of EEG electrodes;
determining from the plurality of signals a state of contact with the user's scalp for each of at least two of the plurality of electrodes; and
displaying on a display a representation of the user's scalp and an indicator of the state of contact for each of the at least two of the plurality of electrodes, each indicator positioned on the representation in a position representative of a position of an associated electrode on the user's scalp.

2. The method of claim 1, wherein displaying the indicator includes selecting one of a plurality of predetermined colors based on the state of contact, and displaying the color in the indicator.

3. The method of claim 2, wherein selecting a color includes selecting green if the state of contact is determined to be satisfactory.

4. The method of claim 2, wherein selecting a color includes selecting red if the state of contact is determined to be unsatisfactory.

5. The method of claim 2, wherein the indicator comprises shape filled with the selected color.

6. The method of claim 5, wherein the shape is a circle.

7. The method of claim 1, wherein determining the state of contact includes determining a numerical indicator of the state of contact.

8. The method of claim 7, wherein the displaying the indicator includes displaying the numerical indicator.

9. The method of claim 8, wherein the numerical indicator is between 0 and 1.

10. The method of claim 1, wherein the indicators are non-overlapping.

11. A method of determining quality of contact of an electrode on a user, comprising:
applying a reference signal to a user;
receiving a measured signal from an electrode for monitoring bioelectric signals of the user; and
determining a state of contact of the electrode with the user based on the measured signal and the reference signal.

12. The method of claim 11, wherein determining the state of contact includes comparing the measured signal to the reference signal.

13. The method of claim 12, wherein comparing the measured signal to the reference signal includes dividing an amplitude of the measured signal by an amplitude of the reference signal to generate a normalized amplitude.

14. The method of claim 11, wherein the reference signal has a frequency greater than 100 Hz.

15. The method of claim 14, wherein the reference signal has a frequency between 200 and 300 Hz.

16. The method of claim 14, wherein the reference signal has an amplitude of 10 uA or less.

17. The method of claim 14, further comprising filtering signals having the frequency from the measured signal to generate a filtered signal and directing the filtered signal to a state detection engine.

18. The method of claim 13, wherein determining the state of contact includes comparing the normalized amplitude to a threshold.

19. The method of claim 18, wherein determining the state of contact includes determining that the state of contact is satisfactory if the normalized amplitude is greater than a value between about 0.7 and 0.9.

20. The method of claim 19, wherein the value is 0.8.

21. The method of claim 13, wherein determining the state of contact includes comparing the normalized amplitude to a plurality of thresholds.

22. The method of claim 11, further comprising displaying on a display an indicator of the state of contact.

23. The method of claim 22, further comprising displaying on the display a representation of the user's scalp, the indicator positioned on the representation in a position representative of a position of the electrode on the user's scalp.

24. A system comprising:
a headset having a plurality of electrodes thereon;
a display; and
one or more processors configured to determine a state of contact of at least two of the plurality of electrodes from signals from the electrodes and displaying an indicator of the state of contact for each of the at least two of the plurality of electrodes on a display.

25. A system comprising:
a headset having a plurality of electrodes thereon;
a reference signal to apply a reference signal to a user wearing the headset; and
a processor configured to determine a state of contact of the plurality of electrodes with the user based on the signal measured from the plurality of electrodes and the reference signal.

26. A computer program product, tangibly stored on machine readable medium, the product comprising instructions operable to cause a processor to:
receive data indicating a state of contact of an electrode with a user's scalp; and
displaying on a display a representation of the user's scalp and an indicator of the state of contact, the indicator positioned on the representation in a position representative of a position of the associated electrode on the user's scalp.

27. A microcontroller configured to determine a state of contact of a plurality of electrodes with the user based on the signal measured from the plurality of electrodes and the reference signal.

28. A computer program product, tangibly stored on machine readable medium, the product comprising instructions operable to cause a processor to:
cause a reference signal to be applied to a user;
receive a measured signal from an electrode for monitoring bioelectric signals of the user; and
determine a state of contact of the electrode with the user based on the measured signal and the reference signal.
